(19) United States
(12) Patent Application Publication
(10) Pub. No.: US 2024/0115276 A1
(43) Pub. Date: Apr. 11, 2024

(54) ARTERIAL PLAQUE TREATMENT SYSTEM

(71) Applicant: Mohammad Reza Rajebi, League City, TX (US)

(72) Inventor: Mohammad Reza Rajebi, League City, TX (US)

(21) Appl. No.: 18/229,304

(22) Filed: Aug. 2, 2023

Related U.S. Application Data
(60) Provisional application No. 63/397,788, filed on Aug. 12, 2022.

(51) Int. Cl. A61B 17/22; A61B 17/00; A61B 17/3207

(52) U.S. Cl. CPC A61B 17/22; A61B 17/00234; A61B 17/3207; A61B 2017/00238; A61B 2017/00292; A61B 2017/00982; A61B 2017/22001; A61B 2017/22038

(57) ABSTRACT

An arterial plaque treatment system includes a drilling member having a proximal end and a distal end, the distal end comprising a drilling mechanism; a locator member having a proximal end and a distal end, the locator member having a lumen sized and shaped to receive the drilling member therein and having a locating mechanism at the distal end, the locating mechanism being moveable between a first position and a second position; and an arterial plaque treatment tool, wherein the locator member is adapted to be advanced in an artery to a position in proximity to an arterial plaque buildup while the locating member is in the first position and then is adapted to position the drilling member at a desired position relative to the arterial plaque buildup when the locating mechanism is moved to the second position. In another version, an atherectomy device is inserted into the arterial plaque through the locator member. A method of treating arterial plaque is also provided.

### PRIORITY

The present application claims the benefit of domestic priority based on United States Provisional Patent Application 63/397,788 filed on Aug. 12, 2022, the entirety of which is incorporated herein by reference.

### BACKGROUND

Atherosclerosis is a disease of the arterial system in which plaque can build up in the wall of an artery. The plaque, which is made of up fat, cholesterol, calcium, and other substances, can result in the narrowing of the artery if untreated. The narrowing of the artery reduces the flow of blood to downstream organs. Atherosclerosis is one of the leading causes of death in the world.

When atherosclerosis becomes severe, surgical treatment is often indicated. One surgical option is vascular bypass surgery whereby blood flow is diverted around a diseased segment of an artery. Less invasive options involve the opening of the artery by removal or displacement of the plaque buildup within the artery. In an atherectomy procedure plaque is removed using a rotating shaver or laser. In an angioplasty procedure, an inflatable balloon is used to open the artery. Sometimes, a mesh tube called a stent will be placed in the artery after removal of plaque to support the walls of the artery.

In an atherectomy procedure, plaque in an artery is pulverized and removed rather than being compressed against the wall of the artery as in angioplasty. Atherectomy is often indicated when the plaque buildup is particularly hard or calcified or when a blockage returns after a prior angioplasty procedure. In a conventional atherectomy procedure, a guidewire is inserted into an artery through a small puncture. The guidewire is threaded through the artery to the site of the blockage. A catheter tipped with a cutting device is inserted along the guidewire so that the cutting device can be located at the blockage. The cutting device may be for example a blade, a rotating drill, or a laser, on the end of the catheter that is used to cut away or vaporize the plaque within the artery.

While effective, conventional atherectomy procedures and systems have some shortcomings and/or potential complications. For example, often the guidewire cannot pass through the plaque, especially when the blockage is calcified and/or blocks a large portion of the artery. Attempts to force the guidewire into the blockage can result in the guidewire being forced into the and potentially through the arterial wall. If a drill or laser is used to create an opening for the guidewire, there is no way to assure the drill or laser is drilling into the plaque, and it can be difficult to maintain the drill or laser at a desired location.

There is therefore a need for an improved arterial plaque treatment system and method. There is further a need for an arterial plaque treatment system and method that can drill or lase into arterial plaque with reduced risk of cutting into an arterial wall. There is still a further need for an arterial plaque treatment system where a drill or laser can be maintained at a desired location.

### SUMMARY

The present invention satisfies these needs. In one aspect of the invention, an improved arterial plaque treatment system is provided.

In another aspect of the invention, an improved arterial plaque treatment method is provided which comprises an improved manner of locating a drilling member, atherectomy device, angioplasty device, or other tool relative to an arterial plaque buildup.

In another aspect of the invention, an arterial plaque treatment system includes a drilling member, such as a rotary drill or laser, and a locator member adapted to position the drilling member at a desired location.

In another aspect of the invention, an arterial plaque treatment system includes a atherectomy device, such as an orbital, rotational, laser, or directional atherectomy device or the like, and a locator member adapted to position the atherectomy device at a desired location.

In another aspect of the invention, an arterial plaque treatment system includes a drilling member, such as a rotary drill or laser, and a locator member such as a drilling position locating member adapted to position the drilling member at a desired location, wherein the drilling member and the positioning member are slidable over a guidewire.

In another aspect of the invention, an arterial plaque treatment system includes a drilling member, such as a rotary drill or laser, and a locating member such as a drilling position locating member adapted to position the drilling member at a desired location, wherein the drilling member and the positioning member are slidable over a guidewire, all of which are receivable within an outer catheter.

In another aspect of the invention, an arterial plaque treatment system includes a drilling member, such as a rotary drill or laser, and a locator member such as a drilling position locating member adapted to position the drilling member at a desired location, wherein the positioning member includes an expandable arterial wall locator at the positioning member distal end.

In another aspect of the invention, an arterial plaque treatment system includes a cutting member, such as a drill or laser, and a locator member such as a cutting member positioning member adapted to position the cutting member at a desired location, wherein the positioning member includes an expandable arterial wall locator at the positioning member distal end, and wherein the expandable arterial wall locator is made of a shape memory alloy.

In another aspect of the invention, an arterial plaque treatment system includes a drilling member, such as a rotary drill or laser, and a locator member such as a drilling position locating member adapted to position the drilling member at a desired location, wherein the positioning member includes an expandable arterial wall locator at the positioning member distal end, and wherein the expandable arterial wall locator is made of nitinol.

In another aspect of the invention, an arterial plaque treatment system includes a drilling member, such as a rotary drill or laser, and a locator member such as a drilling position locating member adapted to position the drilling member at a desired location, wherein the arterial plaque treatment system further includes an arterial plaque treatment device, such as an atherectomy or angioplasty catheter.

In another aspect of the invention, an arterial plaque treatment method includes providing a drilling member, such as a rotary drill or laser, and a locator member such as a drilling position locating member adapted to position the drilling member at a desired location, using the drilling position locating member to position the drilling member at a desired location.

In another aspect of the invention, an arterial plaque treatment method includes providing arterial plaque treatment tool, such as a drilling member, an atherectomy device, or an angioplasty device, and a locator member adapted to position the arterial plaque treatment tool at a desired location, and using the locator member to position the arterial plaque treatment tool at a desired location.

In another aspect of the invention, an arterial plaque treatment system comprises a drilling member having a proximal end and a distal end, the distal end comprising a drilling mechanism; a locator member having a proximal end and a distal end, the locator member having a lumen sized and shaped to receive the drilling member therein and having a locating mechanism at the distal end, the locating mechanism being moveable between a first position and a second position; and an arterial plaque treatment tool, wherein the locator member is adapted to be advanced in an artery to a position in proximity to an arterial plaque buildup while the locating member is in the first position and then is adapted to position the drilling member at a desired position relative to the arterial plaque buildup when the locating mechanism is moved to the second position.

In another aspect of the invention, an arterial plaque treatment system comprises a drilling member having a proximal end and a distal end, the distal end comprising a drilling mechanism; a locator member having a proximal end and a distal end, the locator member having a lumen sized and shaped to receive the drilling member therein and having a locating mechanism at the distal end, the locating mechanism being moveable between a first position and a second position; and an arterial plaque treatment tool, wherein the locator member is adapted to be advanced in an artery to a position in proximity to an arterial plaque buildup while the locating member is in the first position and then is adapted to position the drilling member at a desired position relative to the arterial plaque buildup when the locating mechanism is moved to the second position, wherein the locating mechanism is an expandable member and wherein the first position is a contracted position and the second position is an expanded position.

In another aspect of the invention, an arterial plaque treatment system comprises a drilling member having a proximal end and a distal end, the distal end comprising a drilling mechanism; a locator member having a proximal end and a distal end, the locator member having a lumen sized and shaped to receive the drilling member therein and having a locating mechanism at the distal end, the locating mechanism being moveable between a first position and a second position; and an arterial plaque treatment tool, wherein the locator member is adapted to be advanced in an artery to a position in proximity to an arterial plaque buildup while the locating member is in the first position and then is adapted to position the drilling member at a desired position relative to the arterial plaque buildup when the locating mechanism is moved to the second position, wherein when the locating mechanism is in the second position, the drilling member is advanceable into the arterial plaque buildup at a desired location to create a hole in the arterial plaque buildup and wherein the arterial plaque treatment tool is then insertable into the hole to treat the arterial plaque.

In another aspect of the invention, an arterial plaque treatment system, the system comprises an arterial plaque treatment tool having a proximal end and a distal end, the distal end comprising an arterial plaque treatment member; and a locator member having a proximal end and a distal end, the locator member having a lumen sized and shaped to receive the arterial plaque treatment tool therein and having a locating mechanism at the distal end, the locating mechanism being moveable between a first position and a second position; wherein the locator member is adapted to be advanced in an artery to a position in proximity to an arterial plaque buildup while the locating mechanism is in the first position and then is adapted to position the arterial plaque treatment tool at a desired position relative to the arterial plaque buildup when the locating mechanism is moved to the second position.

In another aspect of the invention, a method of treating arterial plaque comprises providing an arterial plaque treatment tool; providing a locator member having a proximal end and a distal end, the locator member having a lumen sized and shaped to receive the arterial plaque treating tool, the locator member having a locating mechanism that is moveable between a first position and a second position; advancing the locator member through an artery with the locating mechanism in the first position to a position in proximity to an arterial plaque buildup; moving the locating mechanism to the second position when the locator member is in proximity to the arterial plaque buildup; and advancing the arterial plaque treatment tool in the lumen of the locator member into the arterial plaque buildup and treating the arterial plaque buildup, wherein when the locating mechanism is in the second position, the lumen of the locator member is positioned at a desired location relative to the arterial plaque buildup.

In another aspect of the invention, a method of treating arterial plaque comprises providing an arterial plaque treatment tool; providing a locator member having a proximal end and a distal end, the locator member having a lumen sized and shaped to receive the arterial plaque treating tool, the locator member having a locating mechanism that is moveable between a first position and a second position; advancing the locator member through an artery with the locating mechanism in the first position to a position in proximity to an arterial plaque buildup; moving the locating mechanism to the second position when the locator member is in proximity to the arterial plaque buildup; and advancing the arterial plaque treatment tool in the lumen of the locator member into the arterial plaque buildup and treating the arterial plaque buildup, wherein when the locating mechanism is in the second position, the lumen of the locator member is positioned at a desired location relative to the arterial plaque buildup, wherein the arterial plaque treatment tool comprises a drilling member and wherein the method further comprises advancing another arterial plaque treatment tool into a hole created by the drilling member.

In another aspect of the invention, a method of treating arterial plaque comprises providing an arterial plaque treatment tool; providing a locator member having a proximal end and a distal end, the locator member having a lumen sized and shaped to receive the arterial plaque treating tool, the locator member having a locating mechanism that is moveable between a first position and a second position; advancing the locator member through an artery with the locating mechanism in the first position to a position in proximity to an arterial plaque buildup; moving the locating mechanism to the second position when the locator member is in proximity to the arterial plaque buildup; and advancing the arterial plaque treatment tool in the lumen of the locator member into the arterial plaque buildup and treating the arterial plaque buildup, wherein when the locating mechanism is in the second position, the lumen of the locator member is positioned at a desired location relative to the arterial plaque buildup, wherein the arterial plaque treatment tool comprises an atherectomy device.

### DRAWINGS

These features, aspects, and advantages of the present invention will become better understood with regard to the following description, appended claims, and accompanying drawings which illustrate exemplary features of the invention. However, it is to be understood that each of the features can be used in the invention in general, not merely in the context of the particular drawings, and the invention includes any combination of these features, where:

FIG. 1A is an exploded schematic side view of a version of an arterial plaque treatment system according to the present invention;

FIG. 1B is an assembled side view of the arterial plaque treatment system of FIG. 1A in a drilling position locating member undeployed configuration;

FIG. 1C is an assembled side view of the arterial plaque treatment system of FIG. 1A in a drilling position locating member deployed configuration;

FIG. 2A is an assembled sectional side view of the arterial plaque treatment system of FIG. 1A in a drilling position locating member undeployed configuration;

FIG. 2B is an assembled sectional side view of the arterial plaque treatment system of FIG. 1A in a drilling position locating member deployed configuration;

FIG. 3A is a sectional side view of the arterial plaque treatment system of FIG. 1A introduced in an artery having an arterial plaque buildup with a drilling position locating member in an undeployed configuration;

FIG. 3B is a sectional side view of the arterial plaque treatment system of FIG. 1A introduced in an artery having an arterial plaque buildup with the drilling position locating member in a deployed configuration;

FIG. 3C is a sectional side view of the arterial plaque treatment system of FIG. 1A introduced in an artery having an arterial plaque buildup with a drilling member drilling into the arterial plaque at a desired location;

FIG. 3D is a sectional side view of the arterial plaque treatment system of FIG. 1A introduced in an artery having an arterial plaque buildup with the drilling member and drilling position locating member being retracted;

FIG. 3E is a sectional side view of the arterial plaque treatment system of FIG. 1A introduced in an artery having an arterial plaque buildup with the drilling member and drilling position locating member retracted and an arterial plaque treatment device introduced into the artery to treat the arterial plaque buildup;

FIG. 4A is an assembled sectional side view of another version of an arterial plaque treatment system in a drilling position locating member undeployed configuration;

FIG. 4B is an assembled sectional side view of the arterial plaque treatment system of FIG. 4A in a drilling position locating member deployed configuration;

FIG. 5A is an exploded schematic side view of another version of an arterial plaque treatment system according to the present invention having an outer catheter;

FIG. 5B is an assembled side view of the arterial plaque treatment system of FIG. 5A in a drilling position locating member undeployed configuration;

FIG. 5C is an assembled side view of the arterial plaque treatment system of FIG. 5A in a drilling position locating member deployed configuration;

FIG. 6A is a sectional side view of the arterial plaque treatment system of FIG. 5A introduced in an artery having an arterial plaque buildup with a drilling position locating member in an undeployed configuration within an outer catheter;

FIG. 6B is a sectional side view of the arterial plaque treatment system of FIG. 5A introduced in an artery having an arterial plaque buildup with the drilling position locating member advanced from the outer catheter in a deployed configuration;

FIG. 6C is a sectional side view of the arterial plaque treatment system of FIG. 5A introduced in an artery having an arterial plaque buildup with a drilling member drilling into the arterial plaque at a desired location;

FIG. 6D is a sectional side view of the arterial plaque treatment system of FIG. 5A introduced in an artery having an arterial plaque buildup with the drilling member, drilling position locating member, and outer catheter being retracted;

FIG. 6E is a sectional side view of the arterial plaque treatment system of FIG. 5A introduced in an artery having an arterial plaque buildup with the drilling member, drilling position locating member, and outer catheter retracted and an arterial plaque treatment device introduced into the artery to treat the arterial plaque buildup;

FIG. 7A is an assembled sectional side view of a first version of the arterial plaque treatment system of FIG. 1A in a drilling position locating member deployed configuration;

FIG. 7B is an assembled sectional side view of a second version of the arterial plaque treatment system of FIG. 1A in a drilling position locating member deployed configuration; and

FIG. 7C is an assembled sectional side view of a third version of the arterial plaque treatment system of FIG. 1A in a drilling position locating member deployed configuration.

### DESCRIPTION

The present invention relates to an arterial plaque treatment system. In particular, the invention relates to the treatment of arterial plaque using a position locator member for drilling into arterial plaque. Although the arterial plaque treatment system is illustrated and described in the context of being useful for atherectomy and/or angioplasty procedures, the present invention can be useful in other instances. Accordingly, the present invention is not intended to be limited to the examples and embodiments described herein.

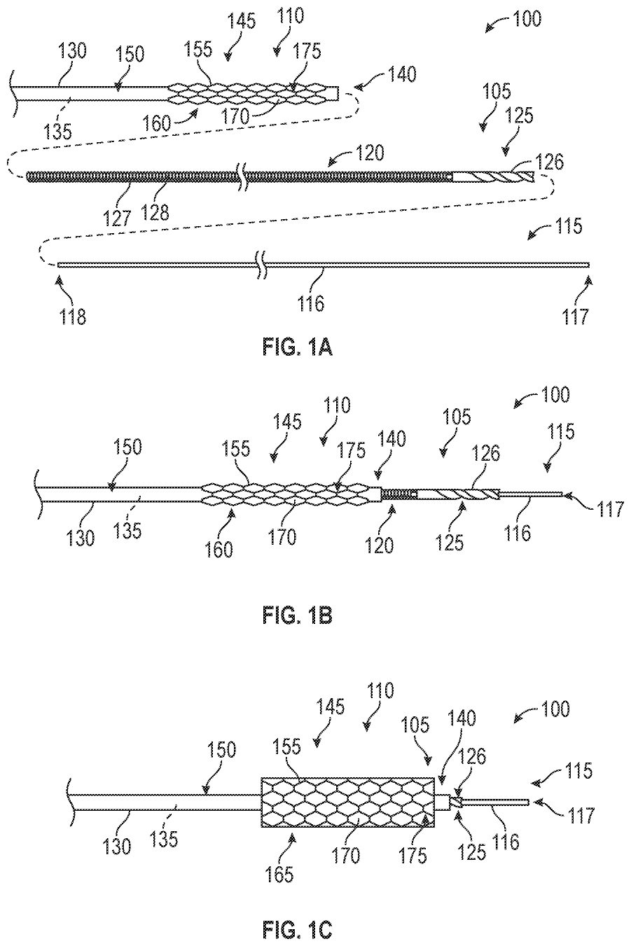

FIGS. 1A, 1B, and 1C show a version of an arterial plaque treatment system 100 of the invention. The arterial plaque treatment system 100 includes a drilling member 105 and a locator member 110 that can be used to position the drilling member 105 or other tool at a desired position during an arterial plaque treatment procedure. The arterial plaque treatment system 100 can be used to treat plaque buildup in an artery in an improved manner over convention treatment systems. By arterial plaque treatment procedure it is meant any procedure that removes, compresses, or otherwise treats an arterial plaque buildup in an artery and/or a procedure that increases the flow through an at least partially blocked artery. Examples of arterial plaque treatment procedures include atherectomy procedures which cut and remove plaque from the wall of an artery and angioplasty procedures which use an expansion device such as a balloon to compress plaque against the wall of the artery. In one version of an arterial plaque treatment procedure according to the invention, a guidewire 115 is inserted into an artery through a small puncture. The guidewire is threaded through the artery to the site of the plaque buildup. The drilling member 105 and the locator member 110 are guided along the guidewire 115 so that the drilling member 105 and the locator member 110 can be positioned at the plaque buildup. The guidewire 115 has a flexible body 116 having a distal end 117 and a proximal end 118. The guidewire 115 can be directed through an arterial system by manipulation of the proximal end 118 in known manner. The guidewire 115 can be any conventional guidewire, such as the HI-TORQUE COMMAND™ from Abbott Laboratories in Chicago, Illinois and the Glidewire Advantage® from Terumo Interventional Systems in Tokyom Japan. Alternatively, the drilling member 105 and the drilling position locating member 110 can be guided into position without a guidewire 115.

The drilling member 105 of the arterial plaque treatment system 100 is a flexible tube or catheter having a flexible body 120 with a drilling mechanism 125 at the drilling member distal end. The drilling mechanism 125 in the version shown is a rotating drill bit 126. Alternatively, the drilling mechanism 125 can be any other type of drilling mechanism, such as a blade or a laser, that is used to cut away or vaporize the plaque within the artery. In the version shown in FIG. 1A, the flexible body 120 is a hollow spring 127 having a lumen 128 extending therethrough for receiving the guidewire 115. Rotation of the flexible body 120 causes rotation of the rotating drill bit 126. Alternatively, the flexible body 120 can be solid and can be guided alongside the guidewire 115 or can be guided on its own without a guidewire 115.

The locator member 110 of the arterial plaque treatment system 100 is a flexible tube or catheter having a flexible body 130 or catheter with a lumen 135 extending from its proximal end to its distal tip 140. The lumen 135 is sized and shaped to receive the drilling member 120 or other tool slidingly therewithin. Located at or near the distal tip 140 is a locating mechanism 145 on the outer surface 150 of the flexible body 130 or catheter. In the version shown, the locating mechanism is an expandable member 155 capable of moving from a contracted position, as shown in FIG. 1A and in the assembled version shown in FIG. 1B, to an expanded position where it expands outwardly away from the outer surface 150 of the flexible body 130, as shown in FIG. 1C. In the contracted position, the locator member 110 is in an undeployed configuration 160 where it may be guided through an artery into a position in proximity to a cite of arterial plaque and/or removed therefrom, and in the expanded position, the locator member 110 is in a deployed configuration 165. The expandable member 155 can be attached or connected to the flexible body 130 or the outer surface 150 of the flexible body 130 in any suitable manner. For example, the ends of the expandable manner 155 can be adhered to the outer surface 150 or a flexible wire or the like can connect the ends of the expandable member 155 to the flexible body 130.

The expandable member 155 may be made of any material or design that allows it to move between its contracted position and its expanded position. In one version, the expandable member is in the form of an at least partially cylindrical sleeve 170 that has a first diameter of its outer surface 175 in the contracted position and a second, larger diameter of its outer surface 175 in the expanded position. In one version, the expandable member 155 is made of a shape memory material, such as a shape memory alloy, such as nitinol, that automatically assumes a memorized shape in response to the removal of force being applied or is response to other changes, such as in response to temperature or the like. For example, in one version, the expandable member 155 can have a memorized shape of its expanded position. The expandable member 155 can be compressed to change its shape into its contracted position, and then when the compressive force is removed, the expandable member 155 will return to its memorized expanded position. In a different version, a temperature responsive shape memory alloy may be used instead or in addition. Shape memory alloys such as nitinol, a nickel-titanium alloy, undergo a phase transformation in their chemical structure when cooled from a stronger, high temperature austenite form to a weaker, low temperature martensite form. When a shape memory alloy is in its martensitic form it is easily deformable. When the alloy is heated beyond its phase transformation temperature, it reverts to it austenite form. In one particular version, the expandable member 155 is a nitinol mesh cylindrical sleeve.

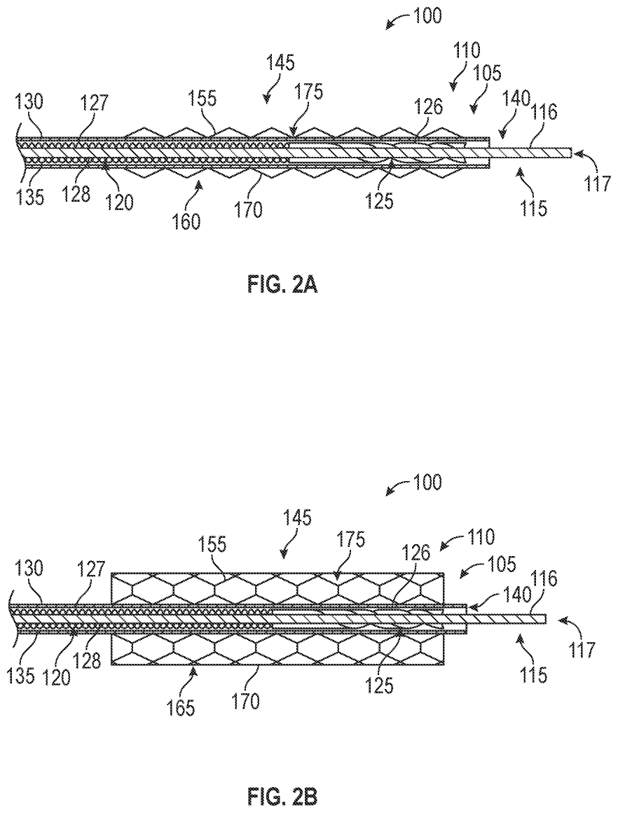

FIGS. 2A and 2B show the arterial plaque treatment system 100 of FIGS. 1A, 1B, and 1C in cross-section in the undeployed configuration 160 and the deployed configuration 165, respectively. These figures show the guidewire 115, if present, slidingly received within the lumen 128 of the drilling member 105 and the drilling member 105 slidingly received within the lumen 135 of the locator member 110.

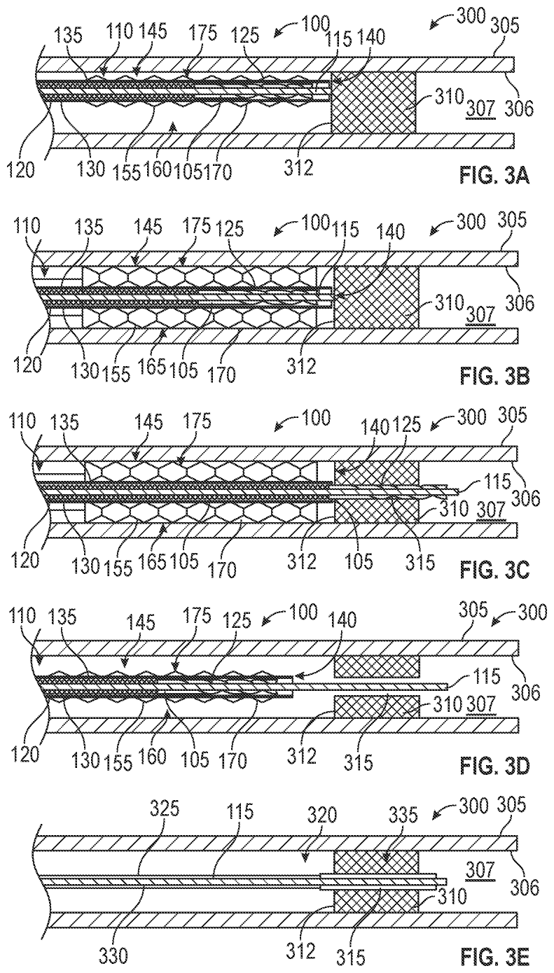

FIGS. 3A through 3E illustrate a method of using the arterial plaque treatment system 100. An arterial system 300 has an artery 305 with an arterial wall 306 that defines an arterial passageway 307 through which blood flows. The artery 305 has an arterial plaque buildup 310 that at least partially blocks the flow of blood through the passageway 307. In the version shown, the arterial plaque buildup is shown as a complete blockage, but the arterial plaque treatment system 100 can be used to treat a partial blockage as well. As shown in FIG. 3A, a guidewire 115 is threaded through the artery until the distal end 117 reaches a first side 312 of the arterial plaque buildup. In the illustration of FIG. 3A, the guidewire 115 is shown as having contacted the arterial buildup near off-center and near one side of the arterial wall 306. The drilling member 105 and the locator member 110 are then guided over or along the guidewire 115 to the arterial plaque 305 with the locator member 110 in the undeployed configuration 160 with the expandable member 155 in the contracted position.

As shown in FIG. 3B, once the drilling member 105 and the locator member 110 are at or near the first side 312 of the arterial plaque buildup 305, the locator member 110 is moved to its deployed configuration 165 by moving the expandable member 155 to its expanded position. The expansion of the expandable member 155 causes the outer surface 175 to contact the arterial wall 306. Because of the symmetry of the expandable member 155, opposing sides of the outer surface 175 of the expandable member 155 are approximately the same distance from the lumen 135 of the locator member 110 and thus the drilling mechanism 125 of the drilling member 105 or other tool. Accordingly, when the expandable member 155 is expanded a sufficient amount so that the outer surface 175 contacts the arterial wall 306, the pressure exerted by the arterial wall 306 on the expanding member 155 will serve to center the locator member 110 within the artery 305.

FIG. 3C shows the drilling member 105 being advanced within the lumen 135 of the locator member 110 and into the arterial plaque 310. Because of the centering of the locator member 110, a drilled hole 315 or opening into at least a portion of, and optionally entirely through, the arterial plaque buildup 310 will be generally at or near the center of the artery 305. By drilling at the desired location, such as the center of the artery 305, there is less risk of drilling into the arterial wall 306. Alternatively, the desired drilling location can be off-center. In such case, the expanded shape of the expandable member 155 can be non-symmetric so that the lumen 135 of the locator member 110, and thus the drilling member 105 or other tool, is positioned at the desired location when the locator member 110 is in the deployed configuration in proximity to the arterial plaque. This version can be useful when arterial plaque buildup 310 is located predominantly on one side of the artery or is otherwise off-center. The locator member 110 can be designed so that the lumen 135 is positioned at or near the center of the arterial plaque buildup 310 or otherwise on the arterial plaque buildup 310.

As shown in FIG. 3D, after the hole 315 is drilled into the desired location in the arterial plaque 310, the locator member 110 is moved to the undeployed configuration 165 by moving the expandable member 155 to its contracted position. The drilling member 105 and the locator member 110 can then be extracted from the artery 305 by sliding them proximally along the guidewire 115 with the guidewire 115 remaining at least partially within the hole 315 in the arterial plaque buildup 310.

Once the drilling member 105 and the locator member 110 are removed from the artery 305 and/or the guidewire 115, an arterial plaque treatment device 320 can be installed onto the guidewire 115 and advanced to the arterial plaque buildup 310. Since the guidewire 115 remains in the hole 315 in the desired location in the arterial plaque buildup 310, the arterial plaque treatment device 320 can be delivered precisely to the desired location, such as the center of the artery 305 where it is safely spaced from the wall 306 of the artery 305. The arterial plaque treatment device 120 has a flexible body 325 with a lumen 330 sized and shaped to receive the guidewire 115. At or near the distal end of the arterial plaque treatment device 320 is an arterial plaque treatment tool 335. The arterial plaque treatment tool 335 can be any tool that removes, compresses, or otherwise treats arterial plaque and/or that increases the blood flow through the artery 305 as a result of the treatment of the arterial plaque buildup 310. For example, in one version, the arterial plaque treatment tool 335 can be an atherectomy tool, such as an orbital, rotational, laser, or directional atherectomy tool that cuts or removes the arterial plaque buildup 310. In another version, the arterial plaque treatment tool 335 can be an angioplasty tool, such as an expandable balloon that expands when in the hole 315 to compress the plaque against the artery wall 306 and thereby increase the size of the opening through the passageway 307.

Prior to the present arterial plaque treatment system 100 of the present invention, it was difficult to drill holes and treat arterial plaque buildup at a desired location. For example, if an unpositioned drill or laser was used to create an opening for the guidewire, there is no way to assure the drill or laser is drilling into the plaque and not doing harm to the artery 305.

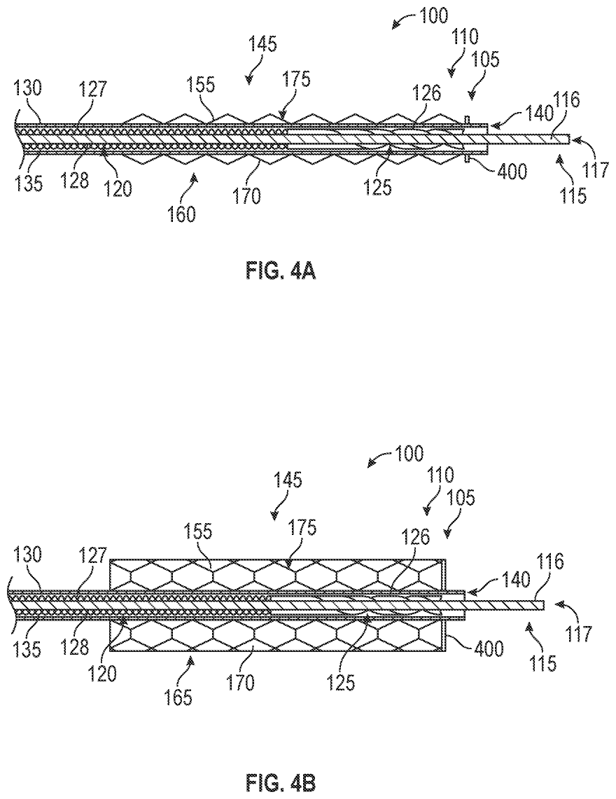

FIGS. 4A and 4B show another version of an arterial plaque treatment system 100 of the invention in cross-section in the undeployed configuration 160 and the deployed configuration 165, respectively. In this version, a layer 400 of material is provided at the distal end of the expandable member 155. In one version, the layer 400 comprises polytetrafluoroethylene (PTFE). The layer 400 can be used to temporarily stop the blood flow though the area of the atherectomy. This will help prevent the migration of small pieces of plaque to smaller arteries distally and thus help prevent them from occluding these arteries.

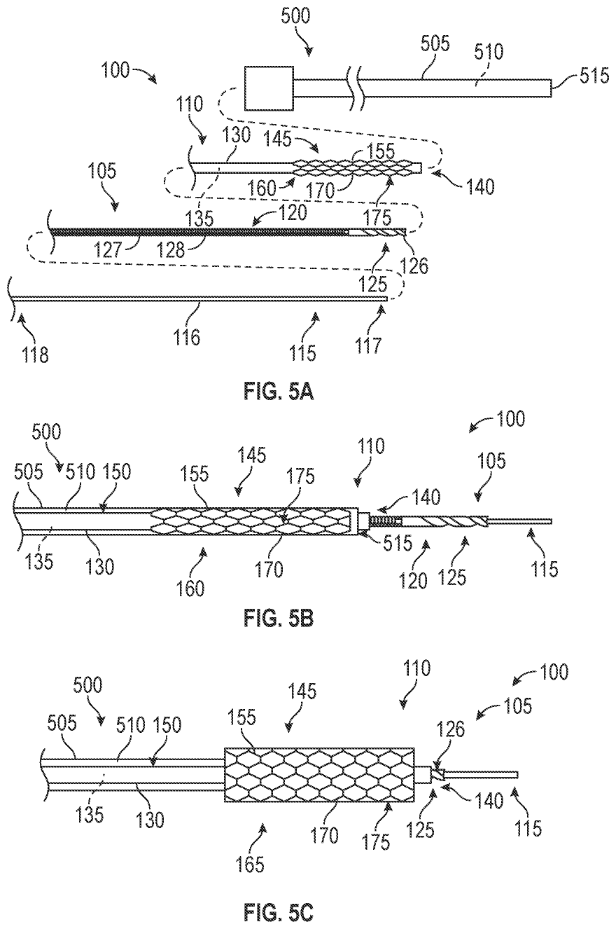

FIG. 5A shows an exploded view of another version of an arterial plaque treatment system 100 of the invention, and FIGS. 5B and 5C show the assembled version in the undeployed configuration 160 and the deployed configuration 165, respectively. In this version, an outer catheter 500 is provided that is capable of housing the drill member 105 and the locator member 110 while they are being guided into position on either the guidewire 115 or in the absence of a guidewire. The outer catheter 500 has a flexible tubular body 505 with a lumen 510 sized and shaped to receive the locator member 110. The lumen 510 is also sized to retain the expandable member 155 when the expandable member 155 is in its compressed position, as can be seen in FIG. 5B. When the locator member 110 is advanced forwardly a sufficient amount that the expandable member 155 is advanced forward of the distal end 515 of the outer catheter, the expandable member 155 may be allowed to expand to its expanded position where the arterial plaque treatment system 100 may be in the deployed configuration 165. The expandable member 155 may be a shape memory device as described above. For example, the expandable member 155 may be an outwardly biased mesh that is biased into its expanded position and may be maintained in its compressed position by being within the outer catheter 500.

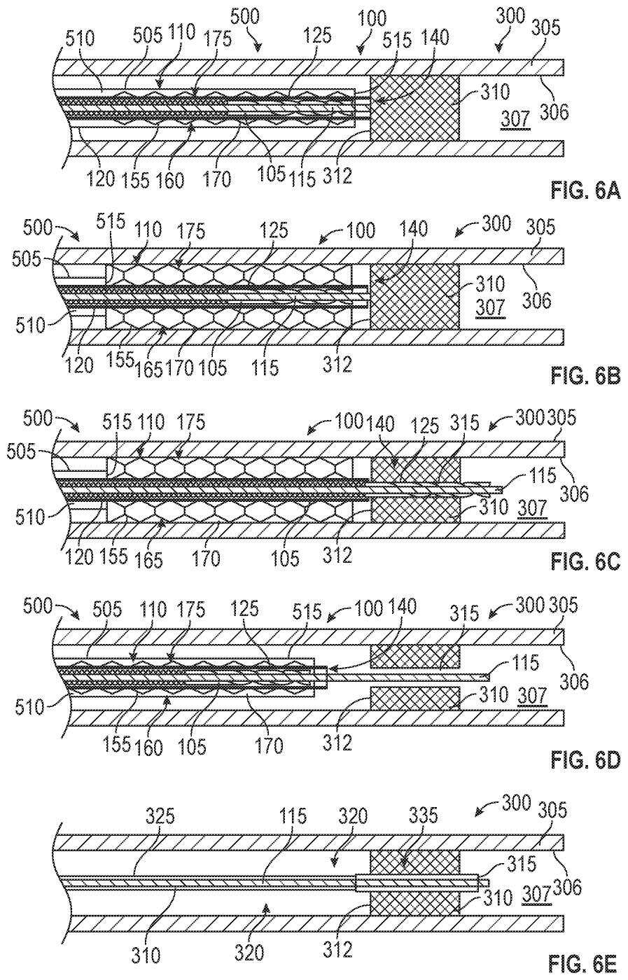

FIGS. 6A through 6E illustrate the operation of the version of the arterial plaque treatment system 100 of FIG. 5A. As can be seen, the steps are similar to the steps of FIGS. 3A through 3E, with the following differences. In FIG. 6A, the outer catheter 500 is also advanced along the guidewire 115 to the arterial plaque buildup 310. In FIG. 6B, the outer catheter 500 is retracted to allow the expandable member 155 to be expanded and the drilling position locator member to be deployed 160. In FIG. 6C, the outer catheter 500 is retracted along with the drilling member 105 and the locator member 110.

In another version, the drilling member 105 itself or another tool can serve as the arterial plaque treatment tool 335. Once positioned by the locator member 110, the drilling member 105 can cut into the arterial plaque in a manner that removes or otherwise treats at least a portion of the arterial plaque. As discussed above, the locator member 110 in this regard can be in the form of a drill bit, a laser, a blade, a scraper, or the like. In one version, the locator member 110 can be considered to be or be replaced with any of the above-mentioned arterial treatment devices, or the like, such as an orbital, rotational, laser, or directional atherectomy tool. Thus, in reference to FIGS. 3A through 3E, in this version, instead of first creating a hole 315 and then inserting an atherectomy device 335 into the hole, the atherectomy device 335 will be inserted directly in the arterial plaque after having been positioned by the locator member 110. In one version in accordance with this version, the atherectomy device 335 can be equipped with a forward end cutting device that effectively serves to create a hole in the arterial plaque into which the atherectomy device can be inserted.

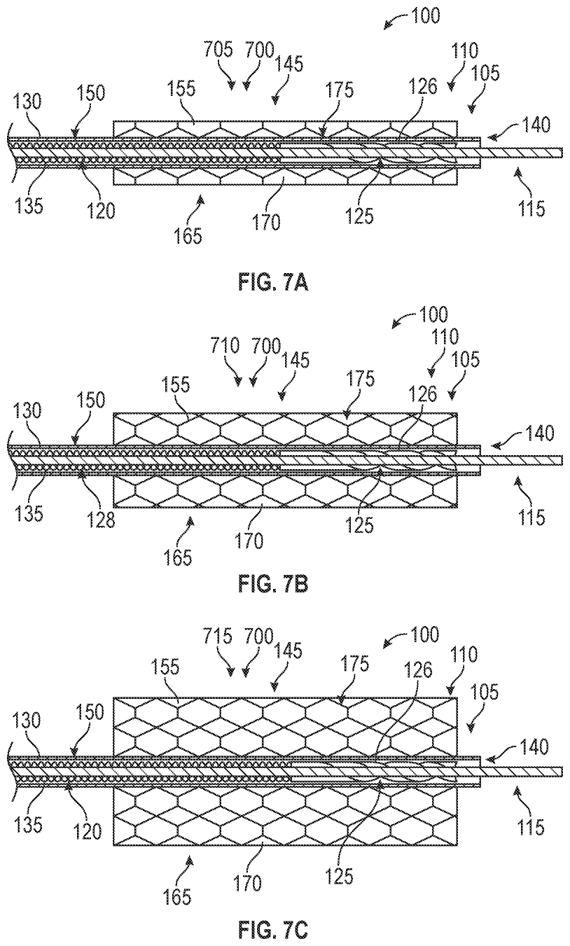

Another version of an arterial plaque treatment system 100 is shown in FIGS. 7A through 7C. In this version, the expandable member 155 is expandable to a predetermined size 700. In FIG. 7A, the expandable member 155 is expandable to a first, relatively small size 705. In FIG. 7B, the expandable member is expandable to a second size, intermediate 710. In FIG. 7C, the expandable member is expandable to a third, relatively large size 7D. The size of the expansion of the expandable member can be selected to correspond to the size of the artery 305 which has the arterial plaque buildup that is to be treated. In one version, the expandable member 155 is configurable to expand to the predetermined size. In another version, a kit of different sized expandable members 155 can be provided so that the surgeon may select the proper size before or during the treatment procedure.

Although the present invention has been described in considerable detail with regard to certain preferred versions thereof, other versions are possible, and alterations, permutations and equivalents of the version shown will become apparent to those skilled in the art upon a reading of the specification and study of the drawings. For example, the cooperating components may be reversed or provided in additional or fewer number, and all directional limitations, such as up and down and the like, can be switched, reversed, or changed as long as doing so is not prohibited by the language herein with regard to a particular version of the invention. Also, the various features of the versions herein can be combined in various ways to provide additional versions of the present invention. Furthermore, certain terminology has been used for the purposes of descriptive clarity, and not to limit the present invention. Throughout this specification and any claims appended hereto, unless the context makes it clear otherwise, the term “comprise” and its variations such as “comprises” and “comprising” should be understood to imply the inclusion of a stated element, limitation, or step but not the exclusion of any other elements, limitations, or steps. Throughout this specification and any claims appended hereto, unless the context makes it clear otherwise, the term “consisting of” and “consisting essentially of” and their variations such as “consists” should be understood to imply the inclusion of a stated element, limitation, or step and not the exclusion of any other elements, limitations, or steps or any other non-essential elements, limitations, or steps, respectively. Throughout the specification, any discussed on a combination of elements, limitations, or steps should be understood to include a disclosure of additional elements, limitations, or steps and the disclosure of the exclusion of additional elements, limitations, or steps. All numerical values, unless otherwise made clear in the disclosure or prosecution, include either the exact value or approximations in the vicinity of the stated numerical values, such as for example about +/− ten percent or as would be recognized by a person or ordinary skill in the art in the disclosed context. The same is true for the use of the terms such as about, substantially, and the like. Also, for any numerical ranges given, unless otherwise made clear in the disclosure, during prosecution, or by being explicitly set forth in a claim, the ranges include either the exact range or approximations in the vicinity of the values at one or both of the ends of the range. When multiple ranges are provided, the disclosed ranges are intended to include any combinations of ends of the ranges with one another and including zero and infinity as possible ends of the ranges. Therefore, any appended or later filed claims should not be limited to the description of the preferred versions contained herein and should include all such alterations, permutations, and equivalents as fall within the true spirit and scope of the present invention.

What is claimed is:

1. An arterial plaque treatment system, the system comprising:
a drilling member having a proximal end and a distal end, the distal end comprising a drilling mechanism;
a locator member having a proximal end and a distal end, the locator member having a lumen sized and shaped to receive the drilling member therein and having a locating mechanism at the distal end, the locating mechanism being moveable between a first position and a second position; and
an arterial plaque treatment tool,
wherein the locator member is adapted to be advanced in an artery to a position in proximity to an arterial plaque buildup while the locating member is in the first position and then is adapted to position the drilling member at a desired position relative to the arterial plaque buildup when the locating mechanism is moved to the second position.

2. An arterial plaque treatment system according to claim 1 wherein the locating mechanism is an expandable member and wherein the first position is a contracted position and the second position is an expanded position.

3. An arterial plaque treatment system according to claim 2 wherein the expandable member is at least partially cylindrical.

4. An arterial plaque treatment system according to claim 2 wherein the expandable member comprises shape memory material so that the expandable member can automatically assume the contracted position or the expanded position.

5. An arterial plaque treatment system according to claim 1 wherein the drilling member comprises a lumen adapted to receive a guidewire.

6. An arterial plaque treatment system according to claim 1 wherein the locator member is adapted to position the locator member lumen substantially in the center of the artery having the arterial plaque buildup.

7. An arterial plaque treatment system according to claim 1 wherein the locator member is adapted to position the locator member lumen substantially off-center in the artery having the arterial plaque buildup.

8. An arterial plaque treatment system according to claim 1 wherein when the locating mechanism is in the second position, the drilling member is advanceable into the arterial plaque buildup at a desired location.

9. An arterial plaque treatment system according to claim 1 wherein when the locating mechanism is in the second position, the drilling member is advanceable into the arterial plaque buildup at a desired location to create a hole in the arterial plaque buildup and wherein the arterial plaque treatment tool is then insertable into the hole to treat the arterial plaque.

10. An arterial plaque treatment system according to claim 1 wherein the drilling member comprises one or more of a rotating drill bit, a laser, and a blade.

11. An arterial plaque treatment system according to claim 1 wherein the arterial plaque treatment tool comprises an atherectomy device.

12. An arterial plaque treatment system according to claim 1 wherein the arterial plaque treatment tool comprises an angioplasty device.

13. An arterial plaque treatment system according to claim 1 further comprising an outer catheter having a lumen sized and shaped to receive the locator member.

14. An arterial plaque treatment system, the system comprising:
an arterial plaque treatment tool having a proximal end and a distal end, the distal end comprising an arterial plaque treatment member; and
a locator member having a proximal end and a distal end, the locator member having a lumen sized and shaped to receive the arterial plaque treatment tool therein and having a locating mechanism at the distal end, the locating mechanism being moveable between a first position and a second position;
wherein the locator member is adapted to be advanced in an artery to a position in proximity to an arterial plaque buildup while the locating mechanism is in the first position and then is adapted to position the arterial plaque treatment tool at a desired position relative to the arterial plaque buildup when the locating mechanism is moved to the second position.

15. An arterial plaque treatment system according to claim 14 wherein the locating mechanism is an expandable member and wherein the first position is a contracted position and the second position is an expanded position.

16. An arterial plaque treatment system according to claim 14 wherein the arterial plaque treatment tool is a drilling member.

17. An arterial plaque treatment system according to claim 14 wherein the arterial plaque treatment tool is an atherectomy device.

18. A method of treating arterial plaque, the method comprising:
providing an arterial plaque treatment tool;
providing a locator member having a proximal end and a distal end, the locator member having a lumen sized and shaped to receive the arterial plaque treating tool, the locator member having a locating mechanism that is moveable between a first position and a second position;
advancing the locator member through an artery with the locating mechanism in the first position to a position in proximity to an arterial plaque buildup;
moving the locating mechanism to the second position when the locator member is in proximity to the arterial plaque buildup; and
advancing the arterial plaque treatment tool in the lumen of the locator member into the arterial plaque buildup and treating the arterial plaque buildup,
wherein when the locating mechanism is in the second position, the lumen of the locator member is positioned at a desired location relative to the arterial plaque buildup.

19. A method according to claim 18 wherein the arterial plaque treatment tool comprises a drilling member and wherein the method further comprises advancing another arterial plaque treatment tool into a hole created by the drilling member.

20. A method according to claim 18 wherein the arterial plaque treatment tool comprises an atherectomy device.
